(19) United States
(12) Patent Application Publication
(10) Pub. No.: US 2006/0142868 A1
(43) Pub. Date: Jun. 29, 2006

(54) Selective crosslinking of orthopaedic implants

(75) Inventor: Brion R. Mimnaugh, North Webster, IN (US)

Correspondence Address: ZIMMER TECHNOLOGY - REEVES, P. O. BOX 1268, ALEDO, TX, 76008, US

(73) Assignee: Zimmer Technology, Inc.

(21) Appl. No.: 11/018,044

(22) Filed: Dec. 21, 2004

(51) Int. Cl. A61F 2/38; B29C 37/02; B28B 11/18; B29C 35/08; B28B 17/00

(52) U.S. Cl. 623/20.32; 623/901; 264/494; 264/138; 264/162

(57) ABSTRACT

A method and starting material are presented for producing property enhancing crosslinking at selected locations within an orthopaedic implant.

### FIELD OF THE INVENTION

The present invention relates to a method for enhancing the mechanical properties of orthopaedic polymers. More particularly, the present invention relates to a method for crosslinking orthopaedic polymers, including ultra-high molecular weight polyethylene (UHMWPE), to increase their wear resistance in orthopaedic bearing applications.

### BACKGROUND

Polymers, including UHMWPE, are commonly used as bearing materials paired with an opposing metal, ceramic, or other component in orthopaedic implants including implants for hips, knees, shoulders, elbows, ankles, vertebral joints, and other locations. Irradiating some polymers, including UHMWPE, can cause changes in their chemical and mechanical properties. The general belief is that the changes in material properties are due to competing reaction pathways, one being crosslinking within and between polymer chains, and another being oxidation. High energy, ionizing radiation, such as gamma or electron beam radiation, breaks molecular bonds, called chain scission, and creates free radicals that are highly reactive species. The severed chains can recombine, crosslink with adjacent chains, or combine with other species such as oxygen.

Crosslinking is known to increase the abrasion resistance of polymers. In orthopaedics it has been indicated as one way to increase the wear life of UHMWPE implants. Crosslinking occurs in polymers when adjacent polymer chains form carbon carbon bonds. Such crosslinking acts to prevent the polymer chains from being pulled or pushed apart. The degree of crosslinking of a material is a function of the radiation dose it receives. While crosslinking may improve some material properties such as wear resistance it may degrade other material properties such as toughness and shear strength.

### SUMMARY

The present invention provides a method and starting material for producing property enhancing crosslinking at selected locations within an orthopaedic implant.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a perspective view of an illustrative implant made according to the present invention;

FIG. 2 is an elevation view of a block of raw material depicting the material being irradiated according to the present invention; and

FIG. 3 is an elevation view of the implant of FIG. 1 illustrating the crosslinked portions of the implant.

### DESCRIPTION OF THE ILLUSTRATIVE EXAMPLES

Polymeric orthopaedic implants may be crosslinked according to the present invention using gamma irradiation, electron beam irradiation, or other suitable forms of irradiation. For example, an electron beam source may be directed toward a sample traveling past the source on a conveyor. Electron beam irradiation of a polymer such as UHMWPE will vary in the depth of penetration depending on the energy level of the accelerated beam. The greater the energy level, the greater the depth of penetration. For example, energy levels can range from 1 to 20 MeV at a beam power of from 1 to 120 kW. Typical commercial electron beam sources use a 10 MeV beam at a beam power of 60 kW. An electron beam of 10 MeV and 60 kW beam power will penetrate UHMWPE to a depth of approximately 4 to 5.5 cm.

The dose range useful for imparting crosslinking with a resultant improvement in wear will vary depending on the application. For example, in a tibial articular surface application, it has been found that a useful dose range is from 3 to 300 kGy, more preferably between 45 and 115 kGy, and still more preferably 45 and 85 kGy.

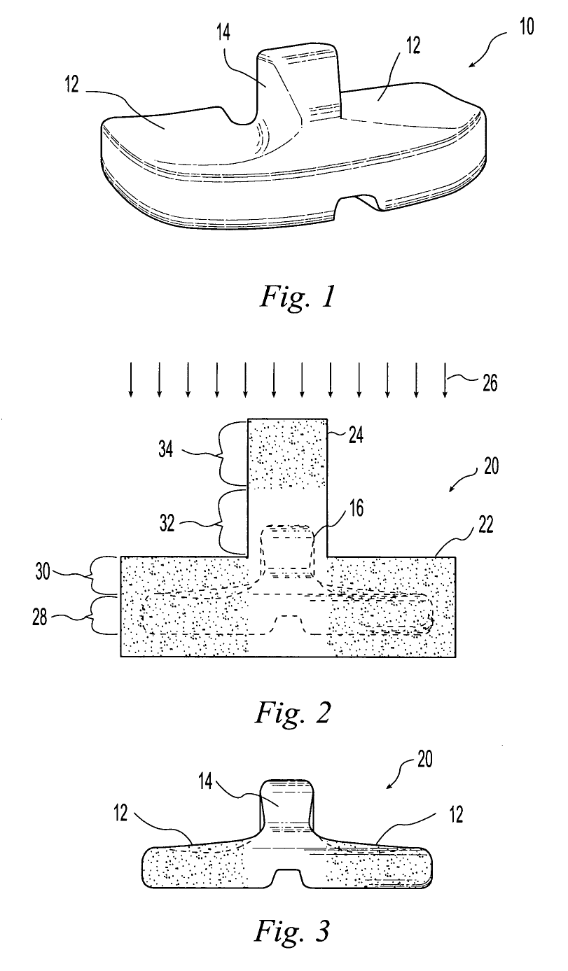

FIG. 1 depicts an illustrative polymeric orthopaedic implant in the form of an UHMWPE tibial knee bearing 10 having condylar articular regions 12 for articulating engagement with a femoral knee component (not shown). The articular regions 12 are crosslinked to increase their wear resistance. The bearing 10 includes a tibial eminence 14 which is lightly crosslinked or non-crosslinked in comparison to the condylar articular regions 12 so that it retains greater toughness and shear strength than the more highly crosslinked articular regions 12.

The implant of FIG. 1 is manufactured by first forming a “T”-shaped UHMWPE blank 20 (FIG. 2) having a horizontal part 22 corresponding to the articular regions 12 and a vertical part 24 corresponding to the tibial eminence 14. The portions of the blank 20 that will be formed into the tibial bearing 10 are indicated by dashed lines 16. The blank 20 is then irradiated along an irradiation axis, as indicated by arrows 26, to crosslink portions of the blank 20. The horizontal part 22 is sized so that the crosslinking radiation penetrates at least into the portion of the blank that will become the surface of the articulating regions 12. The horizontal part includes a first portion 28 sized to accommodate formation of the condylar articular regions 12 and a second portion 30 overlying the first portion 28. The second portion 30 has a thickness along the irradiation axis less than the penetration depth of the crosslinking radiation. The thickness of the second portion 30 may range from zero to slightly less than the penetration depth of the crosslinking radiation. In the illustrative example of FIG. 2, the second portion 30 is sized so that the crosslinking radiation penetrates and causes crosslinking throughout the first portion 28 and consequently throughout the articulating region 12 as indicated by a stippling pattern. The vertical part 24 is sized so that the portion that will become the tibial eminence 14 is at least partially shielded to reduce the amount of crosslinking. The vertical part 24 includes a third portion 32 sized to accommodate formation of the tibial eminence 14 and a fourth portion 34 overlying the third portion 32. The fourth portion 34 has a thickness along the radiation axis relatively greater than the thickness of the second portion 30 so that the third portion 32 is less crosslinked than the first portion 28 by the crosslinking radiation. In the illustrative example of FIG. 2, the fourth portion 34 has a thickness greater than the penetration depth of the crosslinking radiation such that no crosslinking, as indicated by the stippling pattern, occurs in the third portion 32 that will become the tibial eminence 14. Thus, the fourth portion 34 forms an extension of the vertical part 24 that projects beyond what is required to form the tibial eminence 14 and that acts as an integral shield to shield the third portion 32 of the vertical part 24 to reduce or eliminate crosslinking of the third portion 28.

After the blank 20 has been irradiated, it is machined or otherwise formed into the insert 10 shape. During the forming process, the extension 26 is removed. The fully formed insert 10, as shown in FIG. 3, has crosslinked articular regions 12 and a non-crosslinked, or lightly crosslinked, tibial eminence 14. The blank 20 may be sized to produce a single insert 10 or it may be in the form of an elongated “T”-shaped beam from which a plurality of inserts 10 may be formed.

Although an example of a method and polymer blank for forming a selectively crosslinked orthopaedic implant have been described and illustrated in detail, it is to be understood that the same is intended by way of illustration and example only and is not to be taken by way of limitation. Accordingly, variations in and modifications to the method and polymer blank will be apparent to those of ordinary skill in the art, and the following claims are intended to cover all such modifications and equivalents.

What is claimed is:

1. A method of forming an orthopaedic implant comprising the steps of:
forming a polymer blank having the general shape of the implant, a portion of the blank being extended to shield the underlying polymer;
irradiating the blank to crosslink the polymer, the extended portion shielding the underlying polymer from the crosslinking irradiation; and
removing the extended portion from the irradiated blank.

2. The method of claim 1 wherein the polymer blank is “T”-shaped with a horizontal part corresponding to a condylar articular region of a tibial knee bearing and a vertical part corresponding to a tibial eminence of the tibial knee bearing and said extended portion is removed from the vertical part.

3. The method of claim 2 wherein the horizontal part includes a first portion sized to accommodate formation of the condylar articular region and a second portion overlying the first portion, the second portion having a thickness along an irradiation axis less than the penetration depth of the crosslinking radiation such that irradiating the blank along the irradiation axis causes crosslinking to occur in the first portion.

4. The method of claim 3 wherein irradiating the blank along the irradiation axis causes crosslinking throughout the first portion.

5. The method of claim 2 wherein the vertical part includes a third portion sized to accommodate formation of the tibial eminence and a fourth portion overlying the third portion, the fourth portion having a thickness along an irradiation axis greater than the penetration depth of the crosslinking radiation such that the fourth portion shields the third portion from crosslinking irradiation along the irradiation axis.

6. The method of claim 2 wherein the vertical part includes a third portion sized to accommodate formation of the tibial eminence and a fourth portion overlying the third portion, the fourth portion having a thickness along the irradiation axis greater than the thickness of the second portion such that irradiating the blank along the irradiation axis results in more crosslinking in the third portion than in the first portion.

7. The method of claim 2 wherein said step of removing the extended portion comprises machining the orthopaedic implant from the irradiated blank to yield an orthopaedic implant having a tibial eminence that is relatively less crosslinked and a condylar articular region that is relatively more crosslinked.

8. The method of claim 2 wherein forming a polymer blank comprises forming the blank in the shape of an elongated “T”-shaped beam forming an orthopaedic implant comprises forming a plurality of implants from the elongated “T”-shaped beam.

9. A polymer blank for providing the base material from which a tibial knee bearing is formed, the tibial knee bearing including condylar articular regions for articular engagement with a femoral component and a tibial eminence, the blank being subject to crosslinking radiation having a predetermined penetration depth along an irradiation axis prior to forming the implant, the blank comprising:
a first part corresponding to the condylar articular regions of the implant, the first part having a first portion sized to accommodate formation of the condylar articular regions and a second portion overlying the first portion, the second portion having a thickness along the irradiation axis; and
a second part, continuous with the first part, the second part having a third portion sized to accommodate formation of the tibial eminence and a fourth portion overlying the third portion, the fourth portion having a thickness along the irradiation axis greater than the thickness of the second portion.

10. The polymer blank of claim 9 wherein the second portion has a thickness that is less than the penetration depth of the crosslinking radiation and the fourth portion has a thickness that is greater than the penetration depth of the crosslinking radiation.

11. The polymer blank of claim 9 wherein the blank comprises an elongated “T”-shaped beam sized to accommodate forming a plurality of tibial knee bearings.

12. A polymer blank for a prosthetic tibial knee bearing, comprising:
a condylar articular region having a first portion and a second portion overlying the first portion, the second portion having a thickness that is less than the penetration depth of crosslinking radiation; and
a tibial eminence continuous with the condylar articular region, having a third portion and a fourth portion overlying the third portion, the fourth portion having a thickness that is greater than the penetration depth of crosslinking radiation, the thickness of the fourth portion being greater than the thickness of the second portion.
